(19) United States
(12) Patent Application Publication
(10) Pub. No.: US 2023/0320858 A1
(43) Pub. Date: Oct. 12, 2023

(54) Implant System

(71) Applicant: Karl Leibinger Medizintechnik GmbH & Co. KG, Mühlheim/Donau (DE)

(72) Inventors: Adem Aksu, VS-Schwenningen (DE); Heiner Wild, Dürbheim (DE); Frank Reinauer, Emmingen-Liptingen (DE); Tobias Wolfram, Dreieich (DE)

(21) Appl. No.: 18/040,453

(22) PCT Filed: Jul. 17, 2021
(86) PCT No.: PCT/EP2021/070064

§ 371 Date: Feb. 3, 2023

(30) Foreign Application Priority Data
Aug. 6, 2020 (DE) 10 2020 209 981.2

(51) Int. Cl. A61F 2/28; A61B 17/80

(52) U.S. Cl. CPC A61F 2/2846; A61B 17/8095; A61F 2002/30062

(57) ABSTRACT

The disclosure relates to an implant system for promoting an osteosynthetic process at a gap between two bone fragments at a bone of an endoskeleton, in particular of a human mammal. In order to have available an implant system of this kind which allows patient-specific bone reconstruction of a bone, in particular an ulna and radius, wherein the gap between the two bone fragments created during the correction can be supplied with a patient-specific, accurately fitting artificial graft, a graft is provided, which can be inserted into the gap between the two bone fragments; an osteosynthesis plate is provided, which can be fastened to the bone and to the graft ; and a connection device is provided for connecting the graft and the osteosynthesis plate.

The present invention relates to an implant system for promoting an osteosynthetic process at a gap between two bone fragments on a bone of an endoskeleton, in particular of a human mammal.

Fractures of bones on the endoskeleton of mammals result in protracted healing processes. The fractures are often stabilized here by attaching plates of an implant system to the respective bone, wherein sections of the bone are fixed in relation to one another. Such a system is disclosed, for example, in EP 1 440 664 B1.

Although it is applicable to any fractures, the present invention and its underlying problem will be explained on the basis of the human wrist.

The human wrist represents an important component for uncomplicated management of many everyday situations. At the same time, this very important joint in particular is affected very often by injuries. Radius and ulna form the bony foundation of the forearm. Both are curved.

Fractures of the radius and the ulna require exact and continuous bony restoration. If incorrectly healed situations arise there, significant problems in function (restricted supination, pronation, tension, and flexion) and pain can thus be the consequence. Moreover, the risk of the later occurrence of joint arthrosis increases due to resulting misalignments in the two radioulnar joints.

Therefore, a correction osteotomy is carried out in the event of incorrectly healed radius/ulnar fractures, this can result in complete elimination of the symptoms. Depending on the geometry, a gap can arise in the reconstruction of radius or ulna due to the correction angle.

In order to enable or accelerate the healing, this gap is presently filled using spongiosa, for example from the iliac crest or also other donor regions. The gap between the two bone fragments can also remain unfilled up to a certain size or can be filled using a synthetic block. The removal from the iliac crest is an additional painful intervention for the patient, which is also subject to risks. Moreover, this filling material has to be brought into the correct shape by sawing and milling intra-operatively. This can only be done with limited accuracy and costs valuable operation time. If the gap between the two bone fragments remains unfilled, this results in a longer healing time, less stability during the healing phase, and moreover possible aesthetic disadvantages. Therefore, the treatment of the gap using an implant which preferably assists the healing is therefore still sought.

It is therefore the object of the present invention to provide an implant system which ensures patient-specific bone reconstruction of a bone, in particular of ulna and radius.

This object is achieved according to the invention by an implant system having the features of claim 1. In the implant system according to the invention, a graft is provided, which is insertable into the gap between the two bone fragments. Furthermore, an osteosynthesis plate is provided, which is configured to be fastened on the bone and on the graft. A connection device for directly connecting the graft and the osteosynthesis plate is furthermore provided. Using such a resorbable or non-resorbable implant system, on the one hand, the incorrectly healed bone or bones can be brought into a defined location in relation to one another. In addition, however, the healing of the gap created between the two bone fragments is assisted by attaching a graft, wherein this is similarly held in position by connection to the osteotomy plate via the connection device. Due to the filling of the gap between the two bone fragments using a graft which is precisely fitted specifically to the patient, improvements can thus be achieved here over the present treatment options, in that the gap between the two bone fragments is anatomically perfectly filled in such a way that a further intervention on the iliac crest or another donor region can be omitted, in addition, protracted molding or trimming of the graft is dispensed with, and it provides additional stability to the reconstruction.

Advantageous refinements of the implant system according to the invention are found in the dependent claims.

In one advantageous refinement of the implant system according to the invention, the osteosynthesis plate can have at least two plate regions, each of which is fastenable on an associated proximal bone section or distal bone section, respectively, of the relevant bone on both sides of the gap between the two bone fragments, so that the bone sections are held in a defined manner in relation to one another using the respectively fastened plate regions.

In another expedient refinement of the implant system, the osteosynthesis plate can be fastened on the relevant bone so it is secure and can be handled well, if the plate regions of the osteosynthesis plate each have at least one fixing element, which fixes the respective plate region on the associated bone section of the bone. Multiple fixing elements can also be provided in each case here in one or more plate regions.

In a further advantageous refinement, for example, the plate regions of the osteosynthesis plate can each be provided with a hole arrangement having at least one hole, through which the at least one fixing element engages in order to be connected in a simple manner to the associated bone section. The design of the respective hole arrangement can be laid out in this case, for example, according to load criteria and can differ in this way from the further hole arrangement or arrangements. The at least one hole of the respective hole arrangement can preferably pass through the cross section of the osteosynthesis plate perpendicularly here, however, other directions are also conceivable.

In a further refinement, the at least one fixing element for fixing the plate regions can advantageously be made resorbable, so that later stressful removals thereof and complications possibly linked thereto can be omitted.

In one expedient refinement, the at least one fixing element can be made of a metallic material, which is accompanied by the required rigidity for the load-bearing stabilization of the fracture or the gap between two bone fragments.

To connect graft and osteosynthesis plate to one another in a stable and durable manner, in another embodiment of the implant system, the connection device can be provided with one or a plurality of holding elements, which hold the graft on the osteosynthesis plate. One or more identical, or also differing holding elements can be provided here.

In an expedient refinement which manages with few parts, a first holding element can be formed here as at least one projection, which projects essentially transversely to the extension direction of the bone from the osteosynthesis plate and encloses the graft at least in sections on its circumference. The projections of the first holding element are not restricted with respect to the number. The projection of the at least one projection essentially transversely to the extension direction of the bone does not exclude here that the projection itself can also have a formation which has curvatures, in order to adapt itself to structures arranged opposite thereto.

In such a refinement, the first holding element can preferably project with two projections at the height of the graft on both sides of the osteosynthesis plate and can at least partially enclose the graft in the tangential direction. Using the two projections, the holding element can hold and guide a graft in a suitable manner without having to infringe on its structure for this purpose.

In such a refinement, the projection or projections of the first holding element can particularly preferably form a form fit at least with a region of that section of the graft to which they are opposite in such a way that the graft can be arranged without tension on the holding element.

In another preferred refinement, which can increase the stability of the implant system according to the invention in the radial direction, the at least one projection can engage with its end facing away from the osteosynthesis plate in a respective recess associated with it on the graft. Multiple projections each engaging in recesses can also be provided here.

In this case, the at least one projection can preferably be provided on its end facing away from the osteosynthesis plate with at least one undercut in such a way that the projection is fixed particularly well in the respective recess. No limits are placed on the at least one undercut here with respect to its shaping.

Another preferred refinement of the implant system is provided with a second holding element formed by one or more screws, nails, pins, or similar elements or a combination thereof, which, protruding from the osteosynthesis plate, engage in the graft and have good load-bearing properties.

In one advantageous refinement, the second holding element can be formed in each case so it is resorbable from a hybrid component, a composite, a ceramic, a metal, or a polymer. The graft itself can be formed so it is resorbable in one preferred embodiment for the fracture treatment, and is to degrade homogeneously for the stabilization, in addition, a good biocompatibility can be provided and the healing can be functionally assisted. This is also sought for the holding elements and is the case in the above-mentioned materials for the holding elements, in particular also the second holding elements.

In this case, for example, polymers used here can consist of derivatives containing sugar such as poly[L-lactide] or the like, which are enzymatically degradable, or of polyhydroxybutyrate (PHB), whereas composites consist of inorganic, organic, or mixed components that are biocompatible and can convert to hydroxyapatite (HA) and can be provided with coatings. In combination with silicon oxide, for example, HA forms a composite of high porosity, good biomechanical properties, and high load limit. Bioceramics also have good biocompatibility and can include, for example, compositions made up of HA and alpha and beta tricalcium phosphates, which can have components that degrade at different speeds, for example. Biodegradable metals, in the case of which mechanical properties are in the foreground, can be, for example, magnesium (Mg), zinc (Zn), or iron (Fe), without this list being complete.

In a further refinement of the implant system, a third holding element can be provided, which engages through the graft and has a closed cross section. This enables more variable fixing of the graft.

In one preferred refinement, the third holding element can be formed having an elongated, flexible structure, which connects the graft to the osteosynthesis plate and engages through two openings of the osteosynthesis plate. In this way, the fixing becomes even more flexible and variable, since the structure of the holding element permits a flexible path within the graft.

In further preferred refinements, the third holding element can also be formed from a resorbable material, in particular, the third holding element can be formed from a resorbable metal or a polymer.

In one advantageous embodiment of the implant system according to the invention, it is associated with a positioning device having at least one guide means, at the proximal end of which at least one template for introducing the gap into the bone is arranged. With the aid of the positioning device, the gap between the two bone fragments may be preplanned in a suitable manner and with the least possible stress specifically to the patient and treatment and may be introduced at the desired point in time into the bone.

In one expedient refinement, the guide means advantageously has at least two arms at its distal end here, the ends of which form preplanned reference points, which define the location of the positioning device with respect to the bone, by which one or more gaps can be produced accurately between two bone fragments to correct fractures.

The above embodiments and refinements may be combined with one another arbitrarily, if reasonable. Further possible embodiments, refinements, and implementations of the invention also comprise combinations which are not explicitly mentioned of features of the invention described above or hereinafter with respect to the exemplary embodiments. In particular, a person skilled in the art will also add individual aspects as improvements or augmentations to the respective basic form of the present invention.

The invention will be explained in more detail hereinafter on the basis of exemplary embodiments in the figures of the drawing. In the figures, some of which are schematic:

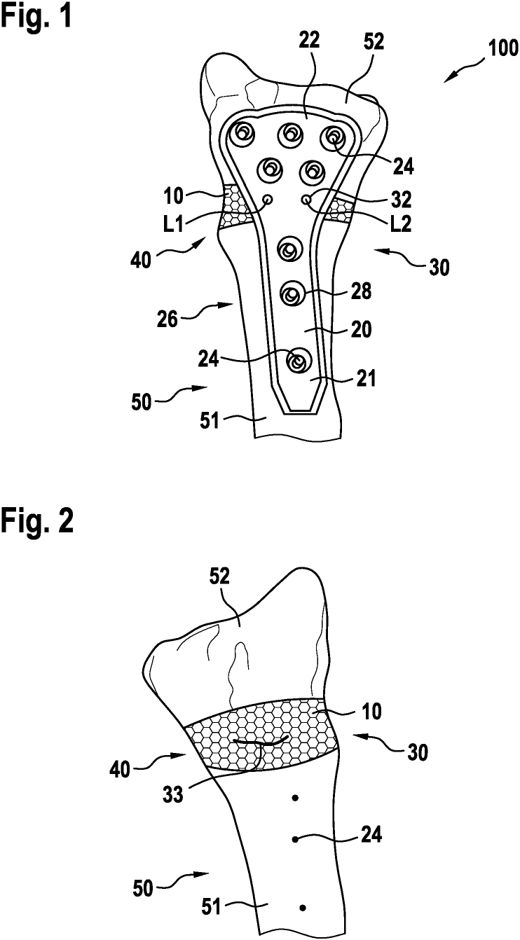

FIG. 1 shows a side view of a first embodiment according to the invention of the implant system having the graft;

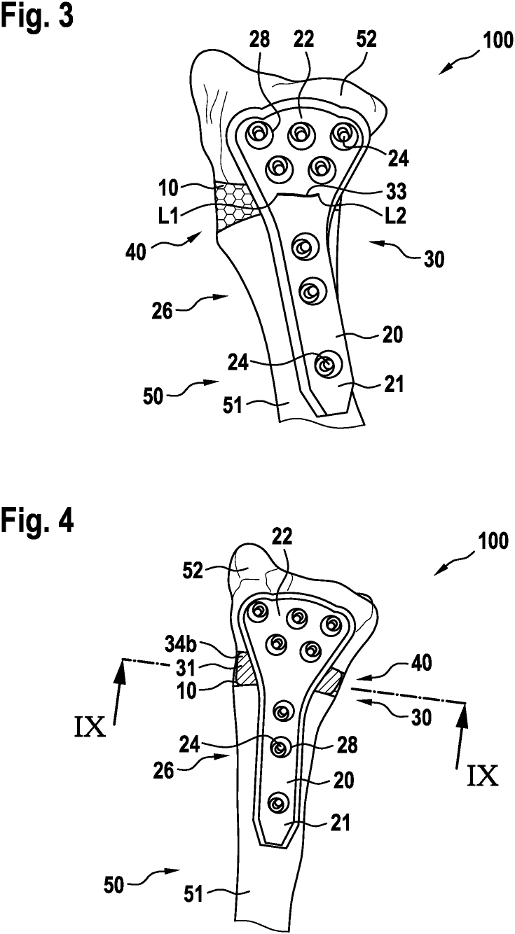

FIGS. 2, 3 show two side views from different viewing angles of a further embodiment according to the invention of the implant system having the graft;

FIGS. 4 to 8 show side views from different viewing angles of further embodiments according to the invention of the implant system having the graft and one or more first holding elements;

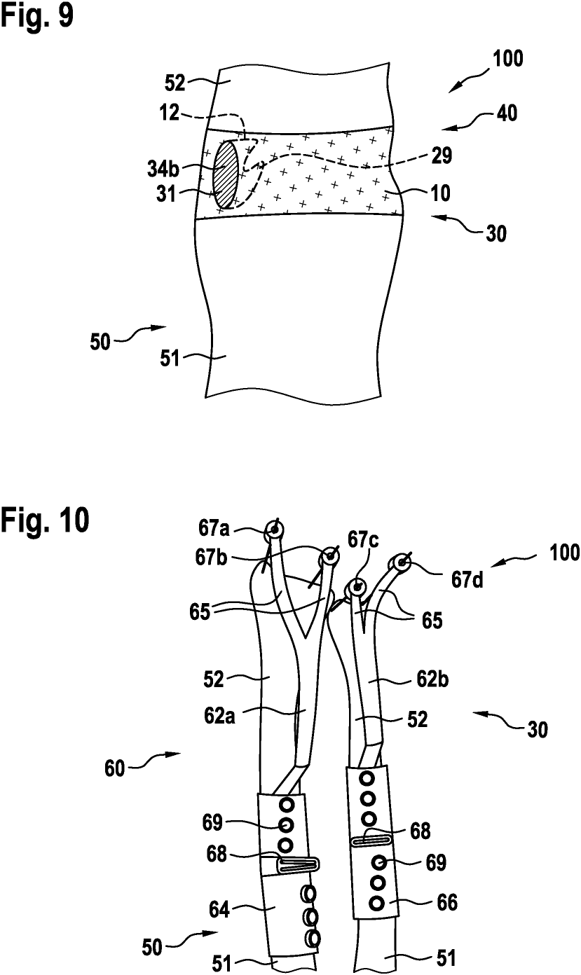

FIG. 9 shows a cross-sectional view of the embodiment from FIG. 4 along line IX-IX; and

FIG. 10 shows a perspective view of a positioning device, associated with the implant system and arranged on the forearm, for introducing a gap between two bone fragments in an ulna bone and a radius bone.

In all figures, identical or functionally identical elements and devices - if not indicated otherwise - have been provided with the same reference signs.

FIG. 1 is a side view of a first embodiment according to the invention of the implant system having the graft.

In FIG. 1, reference sign 100 designates an implant system for promoting an osteosynthetic process at a gap between two bone fragments, for example, an osteotomy gap 40, on a bone 50 of the endoskeleton of a human mammal. A graft 10 is inserted precisely fitted here into the gap previously introduced in a planned manner into the bone between the two bone fragments 40, and an osteosynthesis plate 20 is fastened, facing toward the observer, on the bone 50 and on the graft 10.

In addition, a connection device 30 for directly connecting the graft 10 and the osteosynthesis plate 20 is provided, which is formed by two second holding elements 32, protruding into the image plane, in the form of screws made of a bioresorbable metal. Upon connection, these elements engage at the height of the graft 10 through two holes L1 and L2 located adjacent to one another in the osteosynthesis plate.

These two second holding elements 32 engage through the osteosynthesis plate 20 and engage in the graft 10. Furthermore, the osteosynthesis plate 20 has two plate regions 21, 22, each of which is fastened on an associated proximal bone section 51 or distal bone section 52, respectively, of the bone 50 on both sides of the gap between the two bone fragments 40, by which the two bone sections 51, 52 are held aligned in a defined manner in relation to one another.

The one plate region 21 extends here with lateral edges parallel to one another essentially along the shaft of the bone 50 at the proximal bone section 51. The other plate region 22 widens corresponding to the planar extension of the head of the bone 50 in the image plane in the distal bone section 52, on the one hand, and, on the other hand, it has an angle in relation to the plane spanned by the plate region 21 of the proximal bone section 51.

The two plate regions 21, 22 of the osteosynthesis plate 20 each have a number of fixing elements 24, which fix the respective plate region 21, 22 on the associated bone section 51, 52 of the bone 50. For this purpose, the plate regions 21, 22 of the osteosynthesis plate 20 are each provided with a hole arrangement 26 having a plurality of holes 28, through which the respective fixing element 24 engages. The fixing elements 24 are likewise screws or also pins, for example.

FIGS. 2 and 3 are two side views from different viewing angles of a further embodiment according to the invention of the implant system having the graft.

The embodiment according to FIGS. 2 and 3 is identical except for the differing holding element 33 for fixing the graft 10 on the osteosynthesis plate 20. FIG. 2 shows for this purpose a side of the bone 50 facing away from the osteosynthesis plate 20, while the illustration of FIG. 3 is similar to that of FIG. 1 except for a slightly different viewing angle and shows a top view of the osteosynthesis plate 20.

The holding element 33 according to FIGS. 2 and 3 is formed here from a resorbable polymer having an elongated, flexible structure like a thread, which connects the graft 10 to the osteosynthesis plate 20 and in turn engages through the two holes L1, L2 of the osteosynthesis plate 20. In addition, the holding element 33 in the form of the thread also engages through the porous structure of the graft 10 and has a closed longitudinal extension here.

FIGS. 4 to 8 are side views from different viewing angles of further embodiments according to the invention of the implant system having the graft and corresponding holding elements.

In FIGS. 4-9, the connection device 30 is provided with holding elements 31, which hold the graft 10 on the osteosynthesis plate 20. FIGS. 4 to 8 show side views from different viewing angles here, while FIG. 9 shows a cross-sectional view along line IX-IX of FIG. 4.

The implant system 100 shown in FIG. 4 is again identical in this case to that according to FIG. 1 except for the differing holding element 31.

The first holding element 31 of the connection device 30 according to FIG. 4 is formed at the height of the graft 10 in the form of projections 34a, 34b which project in relation to the observer transversely to the extension direction of the bone 50 to the left from the osteosynthesis plate 20 and enclose the graft 10 on its circumference at least in sections.

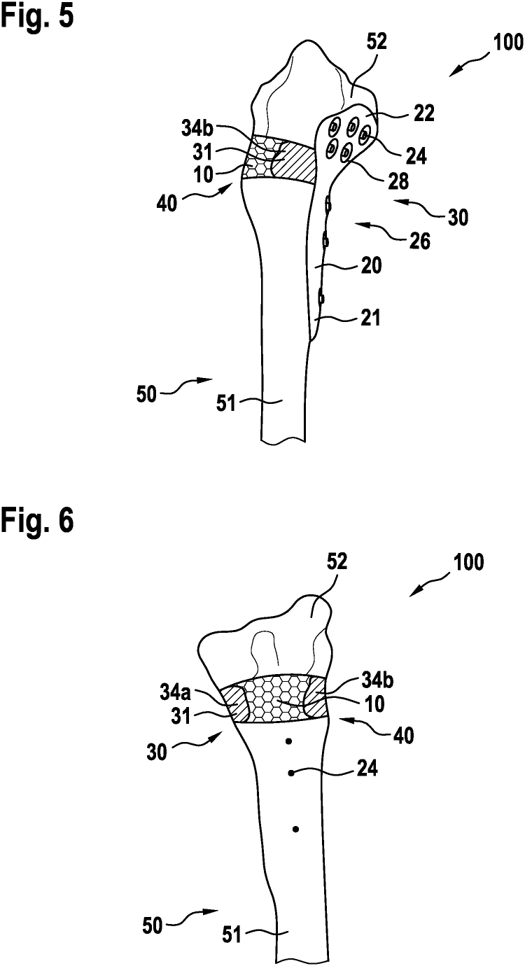

This is apparent in FIG. 5, in which the bone 50 having the osteosynthesis plate 20 and the holding element 31 is rotated 90° counterclockwise around the longitudinal axis of the bone 50.

FIG. 6 shows that the holding element 31 having the two projections 34a, 34b at the height of the graft 10 projects on both sides of the osteosynthesis plate 20 essentially transversely to the extension direction of the bone 50 and partially encloses the graft 10 in the circumferential direction, wherein the free ends of the projections 34a, 34b are apparent in this illustration. The graft 10 is in this way fixed laterally by the projections and axially by the bone regions 51, 52. The projections 34a, 34b of the first holding element 31 form a form fit here at least with a region of that section of the graft 10 to which they are opposite.

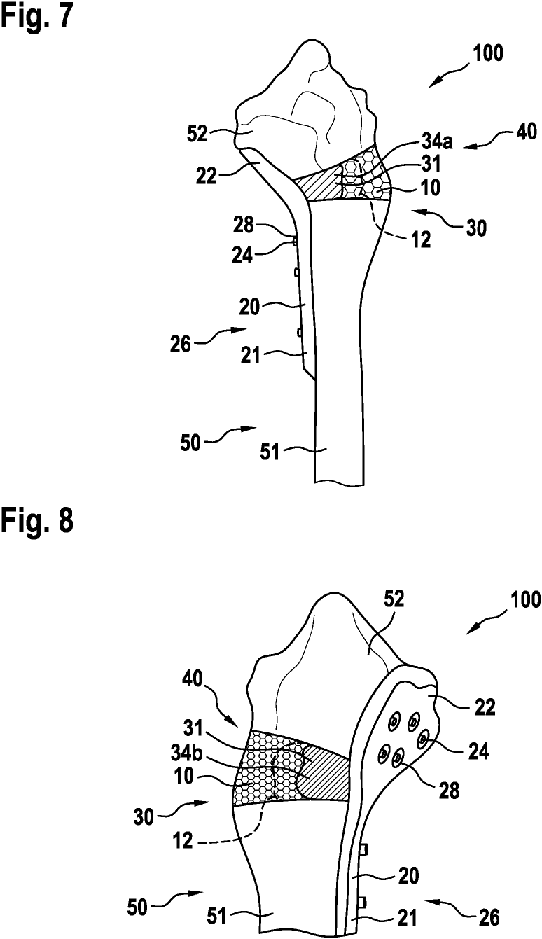

The bone 50 is rotated by a further 90° around the longitudinal axis in FIG. 7, wherein the embodiment shown there furthermore differs from that from FIG. 6 in that the projections 34a, 34b each engage with their end facing away from the osteosynthesis plate 20 in a recess 12 respectively assigned thereto on the graft 10, wherein only the projection 34a is shown in FIG. 7.

In FIG. 8, an implant system shown in FIGS. 4 and 5 can be seen in an illustration having the projection 34b as a holding element 31, which engages here with its end facing away from the osteosynthesis plate 20 in a recess 12 assigned thereto on the graft 10.

FIG. 9 is a cross-sectional view of the embodiment from FIG. 4 along line IX-IX.

It can be seen in the sectional illustration of FIG. 9 that the projection 34b therein of the holding element (not visible in FIG. 4), on the one hand, engages with its end facing away from the osteosynthesis plate 20 in a recess 12 assigned thereto on the graft 10, on the other hand, the projection 34b is provided on its end facing away from the osteosynthesis plate 20 with at least one undercut 29.

FIG. 10 is a perspective view of a positioning device, which is associated with the implant system according to the invention and is arranged on the forearm, for introducing a gap between two bone fragments 40 in an ulna bone and a radius bone.

In the perspective view according to FIG. 10, the positioning device 60 for introducing the gap between the two bone fragments 40 in an ulna bone and a radius bone is shown in the state arranged on the bone sections 51, 52, wherein guide means 62a, 62b are assigned to the positioning device 60, at the proximal ends of which resection templates 64, 66 are arranged for introducing osteotomy gaps 40 (not shown here) into the bone 50. The guide means 62b, 62b each have two arms 65 at their distal ends here, which form predefined reference points 67a-67d, which define the location of the positioning device 40 with respect to the bone 50, and which are held in a clear position by means of holding means (not shown in greater detail), for example, in the form of K-wires at the distal end of the respective bone 50.

The desired osteotomies in the form of the osteotomy gaps 40 can be indicated by saw slots 68 for exact resection for typical saws or also by multiple K-wire guides 69. In the case of these, pre-perforation is performed by multiple screwed-in K-wires (not shown) in the osteotomy, subsequently mobilization is performed by small bone chisels (also not shown). Not only straight, but also curved gaps can be implemented here between two bone fragments 40. In addition, there is also the advantage that only minimal parts of the bone 50 have to be removed.

Exact positioning of the resection templates 64, 66 in the region of the bone 50 to be provided with the gap between the two bone fragments 40 occurs without tilting and stably in punctiform or linear contact in the respective region, so that such templates 64, 66 are to be attached securely and clearly, possibly even without assistance by navigation systems for positioning. In this way, a precise bone resection along the borders of the graft 10 is possible and ensures its good fitting accuracy.

Although the present invention was described above on the basis of preferred exemplary embodiments, it is not restricted thereto, but rather is modifiable in a variety of ways. In particular, the invention may be changed or modified in manifold ways without deviating from the core concept of the invention.

In particular, a bioactive material, for example bioink, can additionally also be provided in or on the graft to accelerate the healing process.

1. Currently amended) An implant system for promoting an osteosynthetic process at a gap between two bone fragments on a bone of an endoskeleton of a mammal, having:
a graft, which is insertable into the gap between the two bone fragments;
an osteosynthesis plate, which is fastenable on the bone and on the graft; and
a connection device for connecting the graft and the osteosynthesis plate.

2. Currently amended) The implant system as claimed in claim 1, wherein the osteosynthesis plate has at least two plate regions, each of which is fastenable on an associated proximal bone section and an associated distal bone section, respectively, of the bone on both sides of the gap between the two bone fragments.

3. Currently amended) The implant system as claimed in claim 2, wherein the plate regions of the osteosynthesis plate each have at least one fixing element which fixes the respective plate region on the associated proximal and distal bone section of the bone.

4. Currently amended) The implant system as claimed in claim 2, wherein the plate regions of the osteosynthesis plate are each provided with a hole arrangement having at least one hole, through which the at least one fixing element can be guided.

5. Currently amended) The implant system as claimed in claim 3, wherein the at least one fixing element is made of a resorbable material.

6. Currently amended) The implant system as claimed in claim 3, wherein the at least one fixing element is formed from a metallic material.

7. Currently amended) The implant system as claimed in claim 1, wherein
the connection device is provided with one or more holding elements which are configured to connect the graft to the osteosynthesis plate.

8. Currently amended) The implant system as claimed in claim 7, wherein a first holding element of said one or more holding elements is formed as at least one projection which projects essentially transversely to the extension direction of the bone from the osteosynthesis plate and encloses the graft on a circumference of the graft at least in sections.

9. Currently amended) The implant system as claimed in claim 8, wherein the first holding element projects with two opposing projections at a height of the graft on opposite sides of the osteosynthesis plate and encloses the graft at least partially in the tangential direction.

10. Currently amended) The implant system as claimed in claim 8, wherein at least one of the at least one projection of the first holding element forms a form fit at least with a region of a section of the graft to which the two opposing projections are opposite.

11. Currently amended) The implant system as claimed in claim 8, wherein the at least one projection engages with an end thereof facing away from the osteosynthesis plate in a recess respectively assigned thereto on the graft.

12. Currently amended) The implant system as claimed in claim 8, wherein the at least one projection is provided on an end thereof facing away from the osteosynthesis plate with at least one undercut.

13. Currently amended) The implant system as claimed in claim 7, wherein a second holding element is formed by one or more fasteners selected from screws, nails, pins and a combination thereof.

14. Currently amended) The implant system as claimed in claim 13, wherein the second holding element is formed a resorbable material selected from a hybrid component, a composite, a ceramic, a metal, and a polymer.

15. Currently amended) The implant system as claimed in claim 7, wherein a third holding element is provided which extends through the graft and has a closed cross section.

16. Currently amended) The implant system as claimed in claim 15, wherein the third holding element includes an elongated, flexible structure which connects the graft to the osteosynthesis plate, wherein the osteosynthesis plate has at least two holes, through which the third holding element can be guided.

17. Currently amended) The implant system as claimed in claim 15, wherein the third holding element is formed from a resorbable material.

18. Currently amended) The implant system as claimed in claim 15, wherein the third holding element is formed from a resorbable metal or a polymer.

19. Currently amended) The implant system as claimed in claim 1, wherein the graft is formed from a resorbable material and/or having a porous structure.

20. Currently amended) The implant system as claimed in claim 1, wherein a positioning device having at least one guide means is assigned to the implant system, at the proximal end of which at least one template is arranged for introducing the gap between the two bone fragments into the bone.

21. Currently amended) The implant system as claimed in claim 20, wherein the at least one guide means has at least two arms at a distal end thereof, the at least two arms having ends which form preplanned reference points, which define the location of the positioning device with respect to the bone.
